(19) United States
(12) Patent Application Publication
(10) Pub. No.: US 2014/0194460 A1
(43) Pub. Date: Jul. 10, 2014

(54) TREATMENT OF ENDOMETRIOSIS

(71) Applicant: Ferring B.V., Hoofddorp (NL)

(72) Inventors: Antonio Pellicer-Martinez, Valencia (ES); Carlos Simon-Valles, Godella-Valencia (ES); Edurne Novella-Maestre, Zaragoza (ES); Joan Carles Arce Saez, Dragor (DK)

(73) Assignee: Ferring B.V., Hoofddorp (NL)

(21) Appl. No.: 13/961,027

(22) Filed: Aug. 7, 2013

Related U.S. Application Data
(63) Continuation of application No. 13/380,098, filed on Mar. 9, 2012, now abandoned.

(30) Foreign Application Priority Data
Jun. 26, 2009 (EP) 09251652.5

(51) Int. Cl. A61K 31/473

(52) U.S. Cl. CPC A61K 31/473
USPC 514/290

(57) ABSTRACT

A pharmaceutical composition comprising quinagolide for the treatment and/or prevention of endometriosis.

The present invention relates to the treatment of endometriosis.

Endometriosis is a chronic gynaecological disease. It may be defined as a presence of endometrial tissue, comprising both glandular epithelium and stroma, outside the uterine cavity. It is a benign gynaecological disorder, which, in a sub-population of female patients, may develop into an aggressive disease. Endometriosis is associated with various distressing symptoms including dysmenorrhoea, dyspareunia, pelvic pain and reduced fertility.

It is known that angiogenesis (the process whereby new blood vessels are formed from pre-existing vessels) may be of importance in the development of endometriosis, and that vascular permeability factor/vascular endothelial growth factor (VPNEGF) has a role in vascular genesis, and in both physiological and pathological angiogenesis. The potential effectiveness of anti-angiogenic therapy for treating endometriosis has been assessed using a study using human endometrial tissues transplanted to immuno-compromised nude mice. Four different anti-angiogenic agents were administered three weeks after the endometrial explants had been transplanted (Nap et al., 2004). All four inhibitors were able to reduce the size of established explants, and new blood vessel formation was stopped. However, the known anti-angiogenic agents are highly toxic, and rather difficult to introduce in a human clinical setting.

It has now been unexpectedly found that a specific dopamine agonist, quinagolide, administered under a specific dosage regime, may provide a particularly effective treatment for endometriosis. The specific dosage regime may provide other benefits, such as improved patient tolerance to quinagolide.

The present invention provides a (pharmaceutical) composition comprising quinagolide for the treatment and/or prevention of endometriosis, wherein the composition is for administration at a dose of 15 to 39 micrograms quinagolide/day for 10 to 20 days (e.g from day 1 to day 15 of treatment); followed by administration at a dose of 40 to 64 micrograms quinagolide/day for a further 10 to 20 days (e.g. for the next 10 to 20 days, e.g. from day 16 to day 30 of treatment); followed by administration at a dose of 65 to 85 micrograms quinagolide/day for at least 2 months, (e.g. for a further 2 to 6 months, e.g. for the next six months, e.g. from day 31 to at least day 87 of treatment). The administration of the dose of 65 to 85 micrograms quinagolide/day (e.g. 75 micrograms quinagolide/day) is continued for at least two months, e.g. for from two to six months, e.g. for 3, 4, 5 months). The administration of the dose of 65 to 85 micrograms quinagolide/day (e.g. 75 micrograms quinagolide/day) may be continued for as long as symptoms persist. The composition may be for the treatment and/or prevention of endometriosis, wherein the composition is for administration at a dose of 25 micrograms quinagolide/day from day 1 to day 15 of treatment; followed by administration at a dose of 50 micrograms quinagolide/day from day 16 to day 30 of treatment; followed by administration at a dose of 75 micrograms quinagolide/day from day 31 to at least day 87 of treatment.

The composition may be for administration for a total duration of treatment of at least 87 days, for example at least 16 weeks, for example at least one year. The composition may be for administration for a total duration of treatment of for example 16 to 22 weeks, for example 18 to 20 weeks.

In a further aspect, the present invention provides a (pharmaceutical) composition comprising quinagolide for the treatment and/or prevention of endometriosis, wherein the composition is for administration at a dose of n micrograms quinagolide/day for 10 to 20 days (e.g from day 1 to day 15 of treatment); followed by administration at a dose of 2n micrograms quinagolide/day for a further 10 to 20 days (e.g. for the next 10 to 20 days, e.g. from day 16 to day 30 of treatment); followed by administration at a dose of 3n micrograms quinagolide/day for at least 2 months (e.g. for a further 2 to 6 months, e.g. for the next six months, e.g. from day 31 to at least day 87 of treatment). The integer n may be, for example, between 15 and 39 (e.g. 16, 17, 18, 19, 20, 21, 22, 23, 24, 25, 26, 27, 28, 29, 30, 31, 32, 33, 34, 35, 36, 37, 38, 39). If n is 25, the quinagolide is administered at a dose of 25 (n) micrograms/day for 10 to 20 days (e.g. 15 days); followed by a dose of 50 (2n) micrograms/day for 10 to 20 days (e.g. 15 days); followed by a dose of 75 (3n) micrograms/day for at least 2 months.

Optionally, the composition may be for administration including at least one step of reducing the dose specified above by 15 to 30 micrograms for a period of 1 to 10 (e.g. 1 to 5) days, during the period of treatment (e.g. during the first 1 to 14 days of treatment). The composition may then be for administration resuming treatment at the point the reduced dose commenced.

The term “dose titration” means the adjustment of (a) the dose of a medication (for example within a dose range) and/or (b) the frequency of the dose (for example within a range of frequency) to provide the desired therapeutic effect. It will be appreciated that the applicants have developed a titrated (adjusted) administration of quinagolide which provides a particularly effective treatment of endometriosis and which may provide other benefits such as improved tolerance to quinagolide.

The term “back titration” means reduction of an (established) dose to a lower effective dose, for example to a dose which is effective to maintain a therapeutic effect established by the initial dose or dose protocol (e.g. that of the invention). In one example, the composition may be for administration at a further (e.g. maintenance) dose of 15 to 64 micrograms quinagolide/day for at least 1 day (e.g. for at least 5 days, e.g. for at least 30 days, e.g. for at least 180 days) after administration of the dose of 65 to 85 micrograms/day. The present applicants have surprisingly found that back titration in this manner after the largest dose of quinagolide may provide e.g. long term prevention of recurrence of endometriosis (and/or symptoms of endometriosis). In an example, a back titration may be used for the prevention or management of non-menstrual pelvic pain or dysmenorrhea. In another example, the composition may be for administration including at least one step of reducing a dose specified in the claims (e.g. reducing one or more of the doses specified in the claims) by 15 to 30 micrograms/day for a period of 1 to 10 (e.g. 1 to 5) days, during the period of treatment (e.g. during the first 1 to 14 days of treatment). The composition may then be for administration resuming treatment at the point the reduced dose commenced. The present applicants have surprisingly found that back titration in this manner may improve patient tolerance.

The present invention provides, in a further aspect, the use of quinagolide in, or in the manufacture of, a (pharmaceutical) composition for the treatment and/or prevention of endometriosis, in which the quinagolide is administered by the methods defined herein.

According to the present invention in a further aspect, there is provided a method of treatment or prevention of endometriosis in a patient in need thereof comprising: a (first) step of administration of a composition comprising quinagolide to the patient at a dose of between 15 and 39 micrograms quinagolide/day for 10 to 20 days (e.g from day 1 to day 15 of treatment); a (second) step of administration of a composition comprising quinagolide to the patient at a dose of between 40 and 64 micrograms quinagolide/day for 10 to 20 days (e.g. for the next 10 to 20 days, e.g. from day 16 to day 30 of treatment); and a (third) step of administration of a composition comprising quinagolide to the patient at a dose of between 65 and 85 micrograms/day for at least 2 months, (e.g. for a further 2 to 6 months, e.g. for the next six months, e.g. from day 31 to at least day 87 of treatment).

The composition may be for the treatment and/or prevention of endometriosis, wherein the composition is for administration at a dose of 25 micrograms quinagolide/day from day 1 to day 15 of treatment; followed by administration at a dose of 50 micrograms quinagolide/day from day 16 to day 30 of treatment; followed by administration at a dose of 75 micrograms quinagolide/day from day 31 to at least day 87 of treatment.

The quinagolide may be administered at a dose of 25 micrograms/day for 15 days; followed by a dose of 50 micrograms/day for 15 days; followed by a dose of 75 micrograms/day for at least 2 months.

The quinagolide may be administered as, for example, a single daily dose; or the daily dose may be divided into two or more (e.g. 3, 4, 5, 6, 7, 98 etc.) sub-doses to be taken at different times over a 24 hour period.

The quinagolide may be administered as a daily dose at the levels above, or as equivalent doses e.g. per week, twice a week, or every two days.

It has been found that administration of (a composition comprising) quinagolide according to the invention to a patient in need thereof may provide substantial clinical benefits such as, for example: significant decrease in the percentage of active endometriotic lesions; significant loss of the cellularity and organisation manifesting characteristics of atrophic or degenerative tissue in endometriotic lesions; and significant decrease in the number of blood vessels in endometriotic lesions. Medicaments based on quinagolide have also the advantage of high dose tolerance, with safe and well documented clinical use records. The use of quinagolide has a further advantage (compared to other treatments for endometriosis) because quinagolide does not inhibit ovulation.

Herein, the term “treatment of endometriosis” includes treatment to reduce (or remove) the amount of endometrial tissue which is present outside the uterine cavity (e.g. reduction or removal of endometriotic lesions); and/or treatment to reduce and/or ameliorate one or more symptoms associated with endometriosis (e.g. treatment to ameliorate and/or reduce the symptoms of dysmenorrhoea; treatment to ameliorate and/or reduce the symptoms of dyspareunia, and/or treatment to ameliorate and/or reduce pelvic pain). The term “treatment of endometriosis” includes treatment to reduce the number of instances, and/or reduce the size of instances, of endometrial tissue which is present outside the uterine cavity (e.g. reduction of number and/or size of endometriotic lesions). The term “treatment of endometriosis” includes treatment to reduce the number of endometrial glands. The term “treatment of endometriosis” includes treatment which results in one or more of: a significant decrease in the percentage of active endometriotic lesions; a significant loss of the cellularity and organisation manifesting characteristics of atrophic or degenerative tissue in endometriotic lesions; and a significant decrease in the number of new blood vessels in endometriotic lesions. The term “treatment of endometriosis” includes treatment to reduce the number and/or size of endometriotic lesions on one or more of the ovary, the uterine cul-de-sac, the uterosacral ligaments, the posterior surfaces of the uterus, the broad ligament, the remaining pelvic peritoneum, the bowel, the urinary tract (including e.g. the bladder, and/or the ureters). The term “treatment of endometriosis” includes treatment and/or management of non-menstrual pain and dysmenorrhea, prevention of recurrence of endometriosis, and prevention of recurrence of non-menstrual pain and dysmenorrhea.

The American Society for Reproductive Medicine (ASRM) defines a classification system for the various stages of endometriosis, dividing this into four stages (stage IV most severe; stage I least severe) [American Society for Reproductive Medicine. Revised American Society for Reproductive Medicine classification of endometriosis: 1996. Fertil Steril 1997; 67, 817 821.]. The term “treatment of endometriosis” includes treatment to reduce the severity of the condition as measured by ASRM classification, e.g. treatment to reduce the severity of the endometriosis from Stage IV, III, II, or I to a lower stage, or until the symptoms are completely alleviated.

The treatment or prevention of endometriosis may be associated with a decrease in the amount of endometrial glands.

The term endometriosis includes, for example, peritoneal endometriosis, ovarian endometriosis and deep endometriosis.

The treatment or prevention of endometriosis may be associated with (or include) management of (improvement of) fertility in a subject having endometriosis (that is, the treatment or prevention of endometriosis may be associated with or include treatment of infertility in a subject having endometriosis).

The treatment or prevention of endometriosis may be in a human or animal subject. The subject may also have, or be prone to, hyperprolactinemia.

According to the present invention in a further aspect there is provided a (pharmaceutical) composition comprising quinagolide for the treatment and/or relief of pain (e.g. pain associated with endometriosis). The composition may be for administration at a dose of 15 to 300 micrograms quinagolide/day, for example 25 to 85 micrograms quinagolide/day.

According to the present invention in a further aspect there is provided a (pharmaceutical) composition comprising quinagolide for the treatment of infertility in a patient having endometriosis. The composition may be for administration at a dose of 15 to 300 micrograms quinagolide/day, for example 25 to 85 micrograms quinagolide/day.

According to the present invention in a further aspect there is provided a (pharmaceutical) composition comprising quinagolide for the treatment and/or prevention of reproductive cancer (for example reproductive cancer associated with for example due to) endometriosis). The reproductive cancer may be cancer of one or more of the reproductive organs, for example one or more of the ovary (ovaries), fallopian tubes, endometrium, cervix, or vagina. The composition may be for administration at a dose of 15 to 300 micrograms quinagolide/day, for example 25 to 85 micrograms quinagolide/day.

According to the present invention in a further aspect, there is provided a method of treatment or relief of pain (e.g. pain associated with endometriosis), and/or a method of treatment of infertility in a patient having endometriosis, and/or a method of treatment of reproductive cancer (for example reproductive cancer associated with for example due to) endometriosis, in a patient in need thereof comprising a step of administration of a composition comprising quinagolide to the patient. The composition may be administered at a dose of 15 to 300 micrograms quinagolide/day, for example 25 to 85 micrograms quinagolide/day.

Quinagolide is (3R,4aR,10aS)-3-(Diethylsulfamoylamino)-6-hydroxy-1-propyl-3,4,4a,5,10,10a-hexahydro-2H-benzo[g]quinoline. The quinagolide is administered as a pharmaceutically acceptable preparation. The composition (comprising quinagolide) may be administered in accordance with the invention in pharmaceutically acceptable compositions that may optionally comprise pharmaceutically acceptable salts, buffering agents, preservatives and excipients. Compositions (e.g. pharmaceutical compositions/preparations) which include quinagolide as active agents are well known in the art and are commercially available. For example, quinagolide is available under the registered trade mark NORPROLAC. The use of such commercially available preparations and compositions in the treatment of endometriosis by the defined methods/administration protocols is according to the invention.

The mode of administration selected will depend on the acuteness and severity of the condition being treated. Any mode of administration that produces desired therapeutic effect without unacceptable adverse effects is relevant in practicing the invention. Such modes of administration may include oral, rectal, topical, transdermal, sublingual, intramuscular, parenteral, intravenous, intracavity, vaginal, and adhesive matrix to be used during surgery. Vaginal administration of the quinagolide is preferred, for example by vaginal pessary or tablet, or by vaginal ring. Various approaches for formulating compositions for use in accordance with the invention are described in the Handbook of Pharmaceutical Excipients, Third Edition, American Pharmaceutical Association, USA and Pharmaceutical Press UK (2000), and Pharmaceutics—The Science of Dosage Form Design, Churchill Livingston (1988).

In a preferred embodiment, the administration is oral. Compositions suitable for oral administration include capsules, cachets, tablets, syrups, elixirs or lozenges.

In an embodiment, the quinagolide is used as the only medical treatment for endometriosis. In other words, the quinagolide may be used in the absence of other medical or surgical treatments [for example, in the absence of danazol].

The administration of quinagolide may be combined with other medical or surgical treatments. The administration of quinagolide may be combined with other medical or surgical treatments for endometriosis, and/or with pain relief medication and/or contraception [for example, administration with NSAIDs and/or hormonal treatments (danazol, OCs, medroxyprogesterone acetate, other progestins, GnRH agonists and antagonists, aromatase inhibitors)]. In a further embodiment, surgical treatment or medical treatment may be used prior to, during or after treatment with quinagolide.

The applicants have found that quinagolide may be administered for long periods of time with therapeutically beneficial effect, and low risk of side effects. The quinagolide may be administered for as long as pain (or other symptom) continues. The patient may be pregnant

The present invention will now be illustrated with reference to the Examples and attached drawings, in which:

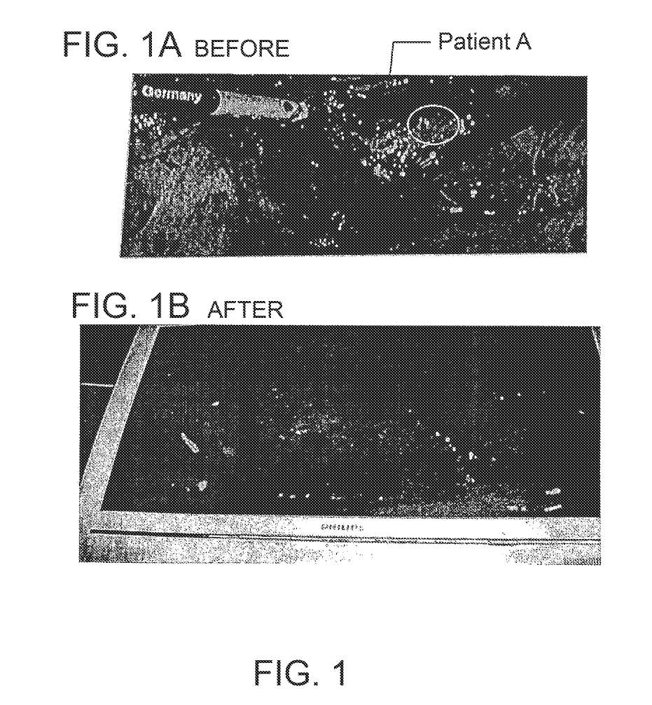

FIG. 1 shows photographs of the marked implant before (FIG. 1A) and after (FIG. 1B) treatment in one subject (“subject A”) treated by the method described below;

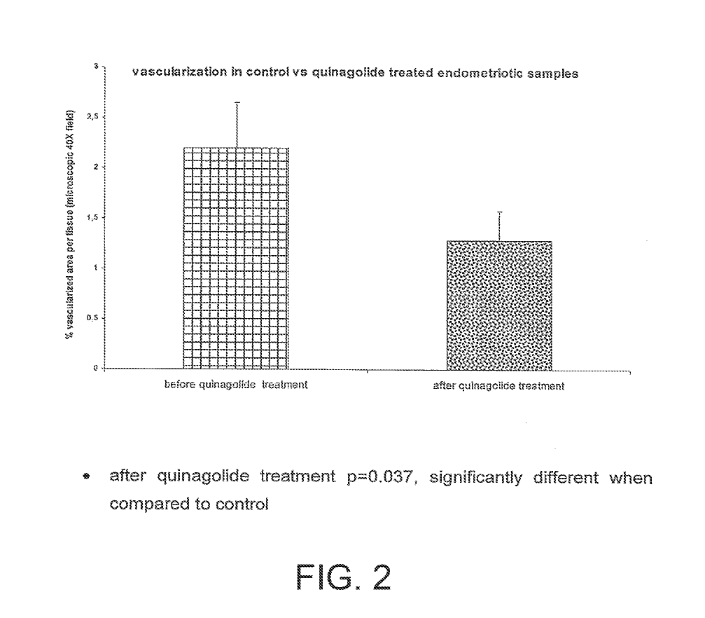

FIG. 2 shows the vascularisation effects of the quinagolide (assessed by immunofluorescence/immunohistochemistry), before and after treatment;

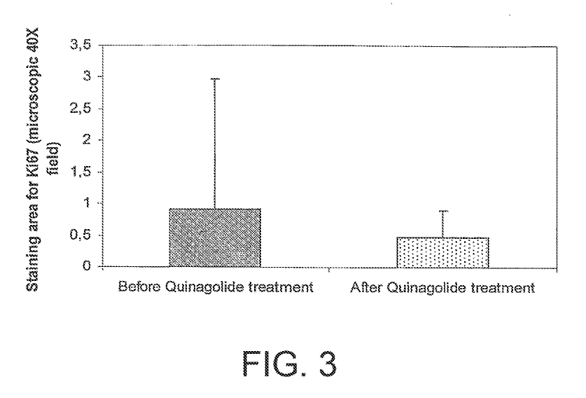

FIG. 3 shows the proliferation of cells before and after treatment, as assessed by Immunocytochemistry with Ki-67 antibody;

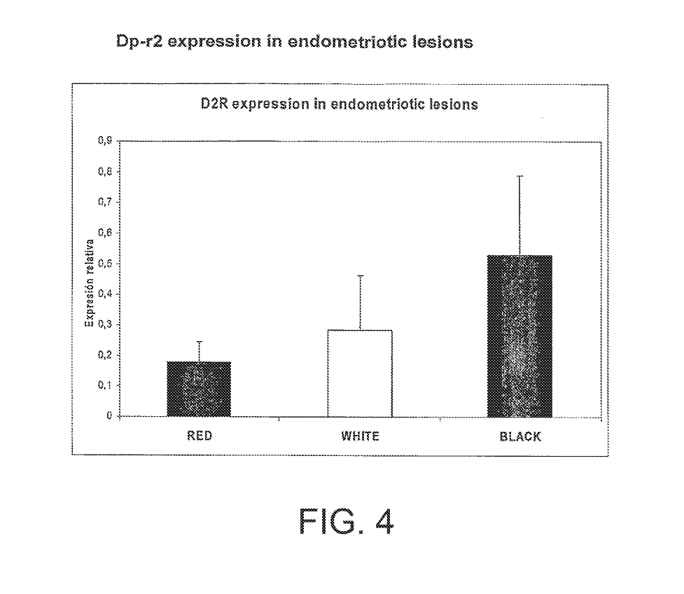

FIG. 4 shows Dp-r2 expression in endometriotic lesions before and after treatment; and

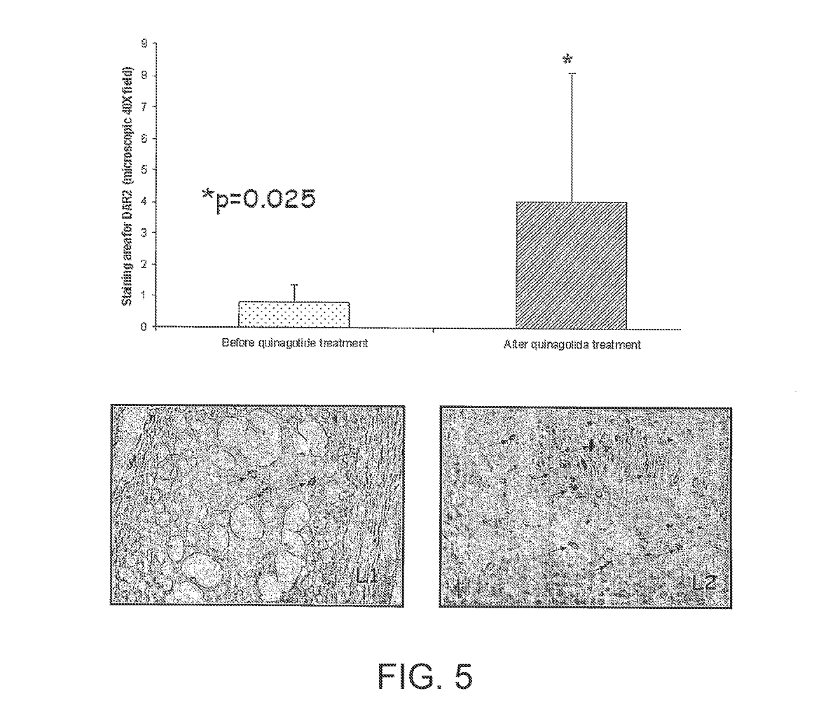

FIG. 5 shows DAR2 expression in endometriotic lesions before and after treatment with quinagolide.

### EXAMPLE 1

### Pilot Study to Test Norprolac in Humans

Approval was obtained to treat nine women with some degree of hyperprolactinemia and concomitant endometriosis. The study was as follows: —

1] Laparoscopy 1: an endometriotic implant was biopsied, and a second endometriotic implant, similar to the biopsied one, was marked with a non-absorbable suture. Pictures were taken before and after surgery from all individuals. The marked implant was photographed (e.g. FIG. 1A). The biopsy sample was analysed as described below. An assessment of ASRM stage pre-treatment was taken. The PRL level (μg/mL) and Ca125 level (IU/ml) for each patient was measured pre-treatment, by known methods.

2] Administration of Norprolac (a pharmaceutical composition comprising quinagolide) according to the following dosage regime:

Stage 1: administration (oral) at a dose (of quinagolide) of 25 micrograms/day for 15 days (i.e. from day 1 to day 15);

Stage 2: administration (oral) at a dose of 50 micrograms/day for 15 days (i.e. from day 16 to day 30);

Stage 3: administration (oral) at a dose of 75 micrograms/day from day 31 for a further four months (up to 5 month total study) or further three months (up to four month total study)

3] Laparoscopy 2: in which the second (marked) implant was biopsied. The marked implant was photographed (e.g. FIG. 1B). The biopsy sample was analysed as described below. A second assessment of the ASRM stage post-treatment was completed. The PRL level (μg/mL) and Ca125 level (IU/ml) for each patient was measured post-treatment, by known methods.

Human Subjects and Study Design

Study Design

To test the concept of anti-angiogenic activity of Drd2-A in patients suffering from endometriosis, this pilot study was conducted in patients with hyperprolactinaemia; a condition for which the use of Drd2-A is indicated. Therefore the subjects were hyperprolactinemic (PRL>30 ng/ml) patients necessitating the administration of a Drd2-A, who simultaneously were suffering from endometriosis and a significant ovarian endometrioma (3 cm or larger) necessitating a first surgical intervention (L1) as well as benefiting from a second look (L2) laparoscopy (high likelihood of adhesion formation). Additional inclusion criteria were: a) open fallopian tubes prior to entrance into the study; b) not having received hormone therapy during the 6 months prior to surgery; c) BMI<22 and finally d) at least four red endometriotic lesions, being at least 2 cm apart from all other lesions had to be present in the cul-de-sac as diagnosed during L1. These red lesions were called the index lesions. This study was approved by the ethics committee of Hospital Universitario Dr Peset. The purpose of it was explained in detail to each subject and informed consent was obtained.

Operative L1 and L2 were performed by two experienced surgeons using modern laparoscopic equipment and the surgeries were recorded by video. The severity of the disease was established according to the Revised American Society for Reproductive Medicine classification of endometriosis: 1966 (16). During L1, 4-6 index peritoneal red lesions were identified before surgical manipulation started. Half of the index lesions were surgically removed and stored as described below. The remaining half index lesions were left behind and marked with an unabsorbable silk knot sature approximately 1 cm away from them. All non-index lesions were subsequently removed and discarded. Out of 12 individuals initially asked to participate, two did not meet inclusion criteria with lack of or insufficient number of red endometriotic lesions in cul-de-sac. Thus the study continued with 10 patients after L1. One patient discontinued treatment.

One week after L1 patients were started on quinagolide (Norprolac, Ferring Pharmaceuticals, Madrid, Spain) in a titrated manner. The starting dose was 25 μg/day during the first 15 days, followed by a dose of 50 μg/day the next 15 days, and finally increased to 75 μg/day onwards during a total treatment period of 18-20 weeks.

During the four month treatment period, i.e. between L1 and L2, patients were monthly monitored in our clinic to assess severity of common side effects of Drd2-A like dizziness, nausea and vomiting. During these visits, blood was drawn for subsequent EIA measurement of serum PRL, a marker of the level of Drd2 activity, to monitor compliance with taking the medication.

At the end of treatment, the L2 was performed and video recorded. During surgery the silk knot was identified in all patients, the suture removed and the area containing the index lesion left behind surgically excised. If significant adhesion formation was detected, lysis of adhesion was performed. The index lesions removed at L1 and L2 were fixed in formalin (approximately 75% of each lesion tissue) for histological verification and descriptive analysis of endometriosis. Formalin fixed samples were also employed for immunohistochemical analysis and subsequent quantification of vascularisation, vessel immatureness, Drd2 expression, cell proliferation, VEGFR2 expression and VEGFR2 activation by techniques well known in the art. The remaining portion of each lesion tissue (25%) was homogenized in Trizol and cryopreserved at −80° C. for subsequent Superarray QF-PCR analysis.

In addition, endometrial tissue was obtained employing a pipelle cannule under anesthesia in L1 and L2. One portion was used for histological and immunohistochemical analysis confirming the proliferative phase of the menstrual cycle and Drd2 expression. The remaining portion was store-frozen at −80° C. for subsequent conventional QF-PCR analysis of Drd2 and VEGFR2 mRNA expression.

Macroscopic Analysis

All laparoscopies were recorded with a Karl Storz (Germany) videocamera. Videorecording was displayed seeking illustrative frames of a blunt metal probe with black or grey cap of known size, in perpendicular position to the lesion. Eligible frames were captured, copied to a PC computer and opened with ImagePro Plus Version 6.03 (Media Cybernetics, Silver Spring, Md., USA) software for image processing. The size of the black or grey cap served as a reference to measure the changes in surface induced by quinagolide at L2 over the lesions left behind during L1.

Analysis of Biopsy Samples

The vascularisation effects of the quinagolide were assessed by immunofluorescence/immunohistochemistry on the biopsy samples taken before and after treatment, by methods well known in the art. The CD31(+) signal was used which specifically stains for vessels; vessels are stained brown. In order to quantify vascularisation at least 10 random 40× microscopic fields per sample were photographed and analysed using Image pro-plus software. Areas of interest corresponding to vessels (brown stain) were segmented and marked; and those corresponding to non vascularised areas were segmented and marked differently. The marked image was then analysed to show the % vascularised area per tissue, and the results for samples before and after treatment are shown in FIG. 2.

Immunocytochemistry with Ki-67 (monoclonal IgG1 DAKO Corp., Denmark) antibody was performed in order to evaluate the proliferative activity of the samples, by methods well known in the art. Ki67 (nuclear staining) is related to proliferative activity of cells The histopathological and subcellular ultrastructural changes were detected using an optical microscope (OM) and/or transmission electronic microscope (TEM) and histochemical staining. The images obtained show proliferating cells as positive for Ki67 with nuclear (brown/black) staining. At least 10 random 40× microscopic fields per sample were photographed and analysed. Areas of interest corresponding to proliferating cells (Ki67+) were segmented and marked, and counted. The marked image was then analysed to show the number of proliferating cells per microscopic field (40+) per tissue, and the proliferation results for samples before and after treatment are shown in FIG. 3.

Dp-r2 expression in human peritoneal endometriotic lesions was measured by methods well known in the art. The relative expression between different types of lesion (red, white, black) is shown in FIG. 4.

DAR2 expression in human endometriotic lesions was measured by staining, by methods well known in the art (FIG. 5, bottom). The marked images were then analysed, and the results for samples before and after treatment with quinagolide are shown in FIG. 5 top.

Results

FIGS. 1A and 1B show photographs of the marked implant before and after treatment in one subject (“subject A”). Macroscopically, a clear effect of Norprolac on peritoneal endometriosis was visible. The feeling of the surgeon who carried out the Laparoscopy procedures was that Norprolac actually decreased the overall endometriosis staging; i.e. significantly reduced the severity of the endometriosis. This was borne out by the ASRM assessment, see below.

FIG. 2 shows the vascularisation effects of the quinagolide (assessed by immunofluorescence/immunohistochemistry), before and after treatment. It can be seen that there is a significant decrease in vascularisation of the endometriotic sample after treatment. Thus, at the microscopic level a significant decrease in the number of blood vessels was found after treatment.

FIG. 3 shows the results of a proliferation study. An immunocyto chemistry study using the Ki-67 antibody (i.e. analysing the degree of cellular proliferation employing antibodies against Ki-67) was used to evaluate the proliferative activity of the implants using methods known in the art. Image counting software was used to count the Ki-67 positive cells and hence calculate the proliferation. A trend towards reduced cell proliferation was observed following treatment with quinagolide.

FIG. 4 shows that inactive endometriotic lesions express higher amounts of Dp-r2 than active lesions. FIG. 5 shows that quinagolide treatment increases the expression of the dopamine receptor 2 (DAR2). These data suggest that quinagolide treatment encourages the manifestation of these dopamine receptors 2.

ASRM Classification

The following Table (Table I) gives the ASRM classification for each patient before and after treatment. In every case, the severity of the endometriosis (as measured by ASRM stage) was reduced. This is a strong indication of the benefit of the treatment.

TABLE I

| | ASRM | ASRM |
|---|---|---|
| Patient | pre-treatment | post treatment |
| 1 | Stage IV | Stage I |
| 2 | Stage IV | Stage II |
| 3 | Stage IV | Stage I |
| 4 | Stage IV | Stage III |
| 5 | Stage IV | Stage II |
| 6 | Stage IV | Stage II |
| 7 | Stage III | Stage I |
| 8 | Stage IV | Stage I |
| 9 | Stage III | Stage I |

Biochemistry

The following Table (Table II) gives the PRL level (μg/mL) and Ca125 level (IU/ml) for each patient before and after treatment. In every case, there was a considerable reduction of the PRL level. In nearly every case, there was a considerable reduction of the Ca125 level. This is a strong indication of the benefit of the treatment.

| | PRL level | | Ca125 level | |
|---|---|---|---|---|
| | (μg/mL) | PRL level | (IU/ml) | Ca125 level |
| | before | (μg/mL) after | before | (IU/ml) after |
| Patient | treatment | treatment | treatment | treatment |
| 1 | 31 | <0.1 | 79.9 | 22 |
| 2 | 20.2 | 6.4 | 39.5 | 24.1 |
| 3 | 32.7 | <0.6 | 45.3 | 20.4 |
| 4 | 102 | 1.1 | 80.6 | 29.9 |
| 5 | 128 | 28.6 | 41.2 | 24.9 |
| 6 | 36 | <0.6 | 24.3 | 25.9 |
| 7 | 91 | 15.1 | 31.5 | 15.5 |
| 8 | 266 | 14 | 45.9 | 18 |
| 9 | 238 | 6.5 | 23.4 | 21.1 |

Macroscopic Appearance and Surface Area of Endometriotic Lesions

All index endometriotic lesions removed during L1 as well as all those left behind and recovered during L2 were of red color. In 2 out of the 9 patients all index endometriotic lesions were absent during L2 suggesting that quinagolide treatment induced a regression of peritoneal endometriotic lesions. Indeed 3 other patients showed vanished lesions combined with lesions still persisting but decreased in size. In the remaining 4 patients, despite all index lesions persisted at L2, all of them but one were reduced in size. Overall out of 23 red index lesions, originally labelled at L1, 8 of them vanished so only 15 were recovered at L2. From those recovered, one was increased in size, another was unchanged and the remaining 13 showed reduction in size when compared to L1. Assuming that vanished lesions represent a 100% size reduction, the mean of the total surface area of index lesions left behind at L1 (36.09±20.56 mm²) for the 9 patients decreased 68% when measured during L2 (11.57±12.54 mm²; p<0.05).

Thus, overall, the quinagolide treatment induced a 68% reduction in size with 35% lesions vanishing after 18-20 weeks treatment. Histological analysis showed signs of tissue degeneration supported by the fact that PAI-1, a potent inhibitor of fibrinolysis, was down-regulated in L2 lession. In addition quinagolide downregulated VEGFNEGFR2 and three proangiogenic cytokines (CCL2, RUNX1 and AGGF) 1 while upreguilating Drd2 and one antiangiogenic cytokine CXCL10 (results not shown).

The results indicate that quinagolide administered at a dose of 25 micrograms/day for 15 days; followed by a dose of 50 micrograms/day for 15 days; followed by a dose of 75 micrograms/day for a further 3 or 4 months, may significantly (visible on a macroscopic scale) reduce the severity of the endometriosis in a human patient. The quinagolide may significantly decrease the blood vessels (vascularisation) in the endometrioic lesions; indicating increased tissue degeneration and hence reduction of the endometriotic tissue on a microscopic scale.

1-19. (canceled)

20. A method of treatment or prevention of endometriosis, treatment or prevention or management of non-menstrual pelvic pain or dysmenorrhea, or prevention of recurrence of endometriosis, in a patient in need thereof comprising:
a step of administration of a composition comprising quinagolide to the patient at a dose of between 15 and 39 micrograms quinagolide/day for 10 to 20 days;
a step of administration of a composition comprising quinagolide to the patient at a dose of between 40 and 64 micrograms quinagolide/day for 10 to 20 days; and
a step of administration of a composition comprising quinagolide to the patient at a dose of between 65 and 85 micrograms/day for at least 2 months.

21. The method of claim 20, wherein the composition comprising quinagolide is administered at a dose of 25 micrograms quinagolide/day from day 1 to day 15 of treatment; followed by administration at a dose of 50 micrograms quinagolide/day from day 16 to day 30 of treatment; and followed by administration at a dose of 75 micrograms quinagolide/day from day 31 to at least day 87 of treatment.

22. The method of claim 20, wherein the patient has, or is prone to, hyperprolactinemia.

23. The method of claim 20, wherein the composition is administered in combination with other surgical or medicinal treatment for endometriosis or pain relief, or in combination with a contraceptive.

24. The method of claim 20, wherein the patient is pregnant.

25. The method of claim 20, wherein the endometriosis is associated with a decrease in the amount of endometrial glands; or associated with reduction or removal of an amount of endometrial tissue which is present outside the uterine cavity; or associated with reduction or amelioration of one or more symptoms associated with endometriosis.

26. The method of claim 20, wherein the composition is administered through vagina.

27. The method of claim 20, wherein the total treatment period is 16 to 22 weeks.

28. The method of claim 27, wherein the total treatment period is 18 to 20 weeks.

29. The method of claim 20, wherein the method further comprises administering a dose of 15 to 64 micrograms quinagolide/day for at least 1 day after administration of the dose of 65 to 85 micrograms/day.

30. The method of claim 20, wherein a specified dose is reduced by 15 to 30 micrograms/day for a period of 1 to 10 days during the period of treatment.

31. A method of treatment or prevention of endometriosis, treatment or prevention or management of non-menstrual pelvic pain or dysmenorrhea, or prevention of recurrence of endometriosis, in a patient in need thereof comprising:
a step of administration of a composition comprising quinagolide to the patient at a dose of n micrograms quinagolide/day for 10 to 20 days;
a step of administration of a composition comprising quinagolide to the patient at a dose of 2n micrograms quinagolide/day for 10 to 20 days; and
a step of administration of a composition comprising quinagolide to the patient at a dose of 3n micrograms/day for at least 2 months;
wherein n is an integer from 15 to 39.

32. The method of claim 31, wherein the composition comprising quinagolide is administered at a dose of 25 micrograms quinagolide/day from day 1 to day 15 of treatment; followed by administration at a dose of 50 micrograms quinagolide/day from day 16 to day 30 of treatment; and followed by administration at a dose of 75 micrograms quinagolide/day from day 31 to at least day 87 of treatment.

33. The method of claim 31, wherein the patient has, or is prone to, hyperprolactinemia.

34. The method of claim 31, wherein the composition is administered in combination with other surgical or medicinal treatment for endometriosis or pain relief, or in combination with a contraceptive.

35. The method of claim 31, wherein the patient is pregnant.

36. The method of claim 31, wherein the endometriosis is associated with a decrease in the amount of endometrial glands; or associated with reduction or removal of an amount of endometrial tissue which is present outside the uterine cavity; or associated with reduction or amelioration of one or more symptoms associated with endometriosis.

37. The method of claim 31, wherein the composition is administered through vagina.

38. The method of claim 31, wherein the total treatment period is 16 to 22 weeks.

39. The method of claim 38, wherein the total treatment period is 18 to 20 weeks.

40. The method of claim 31, wherein the method further comprises administering a dose of 15 to 64 micrograms quinagolide/day for at least 1 day after administration of the dose of 3n micrograms/day.

41. The method of claim 31, wherein a specified dose is reduced by 15 to 30 micrograms/day for a period of 1 to 10 days during the period of treatment.

42. A method of treatment or relief of pain, treatment of infertility in a patient having endometriosis, or treatment of reproductive cancer, in a patient in need thereof, comprising a step of administration of a composition comprising quinagolide to the patient.

43. The method of claim 42, wherein the method is for the treatment or relief of pain.

44. The method of claim 43, wherein the treatment or relief of pain is associated with endometriosis.

45. The method of claim 42, wherein the method is for the treatment or prevention of reproductive cancer.

46. The method of claim 45, wherein the method is the reproductive cancer is associated with endometriosis.

47. The method of claim 42, wherein the method is for the treatment of infertility in a patient having endometriosis.

48. The method of claim 42, wherein the composition comprises 15 to 300 micrograms quinagolide.
